(19) United States
(12) Patent Application Publication
(10) Pub. No.: US 2020/0170626 A1
(43) Pub. Date: Jun. 4, 2020

(54) DEVICE FOR PREPARING A SAMPLE TO BE ANALYZED AND METHOD FOR PREPARING A SAMPLE TO BE ANALYZED

(71) Applicant: GAUDLITZ GMBH, Coburg (DE)

(72) Inventor: Walter FRANZ, Gemünda (DE)

(21) Appl. No.: 16/624,971

(22) PCT Filed: Jun. 22, 2018
(86) PCT No.: PCT/EP2018/066818

§ 371 Date: Dec. 20, 2019

(30) Foreign Application Priority Data
Jun. 23, 2017 (DE) 10 2017 114 032.8

(51) Int. Cl. A61B 10/00

(52) U.S. Cl. CPC A61B 10/0038; B01L 3/502; A61B 10/0096

(57) ABSTRACT

The invention relates to a device for the preparation of a sample to be analysed, in particular a stool sample, comprising a first container which has a first container opening and forms a receiving chamber for a suspension liquid in which the sample is at least partially contained, a second container which has a second container opening and forms a second receiving chamber, wherein the first container is at least partially receivable or received in the second receiving chamber, and a sample holder for insertion into the first container opening. The invention is characterised in that a liquid outlet is formed in the first container, and in that the first container and the second container are formed such that suspension liquid passes from the first receiving chamber into the second receiving chamber when the first container is moved out of the second receiving chamber. Furthermore, the invention relates to a method for preparing a sample to be analysed.

The present invention relates to a device for the preparation of a sample to be analysed, in particular a stool sample, comprising a first container which has a first container opening and forms a receiving chamber for a suspension liquid in which the sample is at least partially contained, a second container which has a second container opening and forms a second receiving chamber, wherein the first container is at least partially receivable or received in the second receiving chamber, and a sample holder for insertion into the first container opening. The invention also relates to a method for preparing a sample to be analysed.

A device for the preparation of a sample to be analysed is known from DE 10 2008 057 866 A1.

The invention has the object to specify a device for the preparation of a sample to be analysed, in particular a stool sample, the handling of which is particularly simple and safe. Furthermore, the invention also has the object to specify a method for the preparation of a sample to be analysed which comprises improved handling.

The task is solved by a device with the characteristics of claim 1. Accordingly, it is provided for the device mentioned at the beginning that a liquid outlet is formed in the first container, and that the first and second containers are formed such that the suspension liquid passes from the first receiving chamber into the second receiving chamber when the first container is moved out of the second receiving chamber. This design makes it particularly easy to prepare a sample. In particular, this design allows the sample to be analysed, such as in particular a stool sample, to be at least partially dissolved in a liquid which may be contained in the first receiving chamber to form the suspension liquid. The first container can then be moved out (in particular pulled out) of the second container, wherein at least part of the suspension liquid passes through the liquid outlet into the second receiving chamber. In doing so, in particular insoluble components can be retained in the first receiving chamber. This is essential for a subsequent analysis in an analysis device. In particular in the case of automated and/or sensitive analysis devices, insoluble components can lead to an impairment of the analysis process. In some cases, insoluble components may also lead to failure of the analysis device. This can be reliably avoided by means of the invention. The handling is simple and unintentional contact with the sample material is avoided. The device allows hygienic handling, in particular for stool samples. In addition, adverse influences on the analysis result can be avoided. Besides the preparation of the sample, the device can also be configured for performing the sample collection (in particular a stool sample).

Moreover, the task is solved by a method for preparing a sample to be analysed, in particular a stool sample, comprising the following steps:
Providing the described device for the preparation of a sample to be analysed, having a first and a second container;
Introducing a liquid into the first receiving chamber;
Placing the sample in the first receiving chamber;
Mixing the sample with the liquid to produce a suspension liquid in the first receiving chamber;
Moving the first container out of the second container, wherein the suspension liquid passes through the liquid outlet from the first receiving chamber into the second receiving chamber.

Further advantageous characteristics of the device and the method are described below, which individually or in any reasonable combination form the object of the invention.

According to an advantageous embodiment of the invention, it is provided that the first and second containers are designed in such a way that, when the first container is moved out of the second receiving chamber, a negative pressure is created at the liquid outlet, so that suspension liquid is conveyed from the first container through the liquid outlet into the second container. In particular, the suspension liquid can be sucked through the liquid outlet in this way.

According to an advantageous embodiment of the invention, it is provided that between the first and the second container a negative pressure chamber is formed which communicates with the liquid outlet. The negative pressure chamber can be sealed against the surroundings in particular by the seal described below. During the pulling out process, the negative pressure chamber receives the liquid that has passed through the liquid outlet.

According to an advantageous embodiment of the invention, provision is made for a seal to be provided which seals the first container against the second container in such a way that, when the first container is pulled out of the second container, a negative pressure is created at the liquid outlet so that the suspension liquid is conveyed from the first container through the liquid outlet into the second container.

According to an advantageous embodiment of the invention, it is provided that the seal comprises at least one circumferential projection arranged on the first and/or the second container. The circumferential projection is preferably arranged on an outside of the first container or on an inside of the second container. The arrangement of the circumferential projection on the outside of the first container is preferred. The circumferential projection may be formed in particular by a radially outwardly projecting portion of the side wall or bottom portion of the first container. Such a portion can be produced by injection moulding during the manufacture of the first body, without any additional assembly work. In addition, in the side wall or in the bottom portion of the first container, an accommodation can be provided for a sealing element (e.g. a sealing ring), which forms the circumferential projection. Such an accommodation is preferably formed to be groove-shaped and circumferential.

According to a particularly preferred embodiment of the invention, it is provided for a filter to be arranged at the liquid outlet. The filter makes it possible to filter the suspension liquid as it passes through the liquid outlet. This is particularly effective in preventing unwanted particles from entering the second receiving chamber. The filter may, for example, contain a filter material containing fibres. Alternatively or additionally, the filter can have at least one filter opening, which can be arranged e.g. in a plastic part. In particular, the filter may be formed by a sieve, e.g. made of plastic.

Preferably, the first container has a side wall and a bottom portion. The liquid outlet can be located in the bottom portion. It is preferred for the first container to have a tubular shape. In particular, the first container is elongated. Preferably, the first container has a circular cross-section.

Preferably, the second container has a side wall and a bottom portion. It is preferred for the second container to have a tubular shape. In particular, the second container is elongated. Preferably, the second container has a circular cross-section. The bottom portion and the side wall of the second container may be in particular formed as one piece.

Preferably, the second container has an inner contour that is slightly larger than an outer contour of the first container. In this way, the first container can be received into the second container in a sliding manner.

Preferably, the first container forms an inner sleeve and the second container an outer sleeve.

In accordance with the invention, the device can comprise a sample holder which is insertable into the first container opening. Preferably, the sample holder has one or more recesses, in which a defined quantity of the sample can be received. Preferably the sample holder is elongated. In particular, the sample holder may be a sample holder rod. The sample holder may have a connecting portion, by means of which it is inserted into a recess of the hand grip element. Depending on the requirements of the analysis, the sample holder may be exchangeable. This means that recesses having an adapted shape and/or volume can be provided in each case.

Preferably, provision is made for a stripping device for the sample holder to be provided on the first container. In particular, the stripping device may be designed as a stripping sleeve. This ensures that only the desired quantity of the sample reaches the first receiving chamber. When the sample holder is inserted into the first receiving chamber, the stripping device can abut the sample holder in such a way that excess sample material is stripped off. In particular, sample material that protrudes beyond the recesses can be stripped off. In doing so, the stripping device preferably forms a calibration hole, which allows an exact dosing of the quantity of the sample introduced into the first receiving chamber. Moreover, an additional seal can be achieved with the stripping device by ensuring that the stripping device sealingly abuts the sample holder. If, prior to the insertion of the sample holder into the first receiving chamber, the diameter of the sample holder is slightly larger than the diameter of the elastically designed stripping device, the stripping device can fit closely around the sample holder in a prestressed manner to provide an improved seal.

Preferably, a closure is provided which closes the container opening of the first container in a liquid-tight manner. In a preferred manner, the closure comprises a cap. The closure preferably comprises a plug which is insertable or inserted into the container opening. The closure may be provided with sealing lips extending in a radial direction which abut an inner side of the first container.

Preferably, the closure has a hand grip element. In particular, the hand grip element can have two opposite gripping surfaces. Then the hand grip element can be held in a particularly easily manner with two fingers.

It is preferable for the closure to have a knurled surface on its outer circumference.

The closure should preferably have a seal that seals the closure with respect to the first container. In particular, the seal may include a conical sealing element.

Preferably, the closure can be placed in a partially open position in which the first receiving chamber is connected to the surroundings when the closure is in place. In this way, air can flow into the first receiving chamber, while the first container is pulled out of the second container and suspension liquid is conveyed from the first to the second receiving chamber.

Furthermore, in order to achieve an effective seal between the first container and the closure, an internal contour on the first container may preferably be formed to have a conical shape. In particular, the first container may have a conical inner contour at the first container opening which forms a sealing edge.

Preferably, it is intended that the closure is connected to the first container via a bayonet catch. It is preferable if the way to move the closure from the closed to an open position of the bayonet is between 30° and 90°, preferably between 50° and 70°. Preferably the bayonet catch has a latching function. For this purpose, the bayonet catch may have bayonet tabs, each with a nose, at one leading edge. This can prevent unintentional opening.

Preferably, the bayonet catch has a recess disposed in a tab of the catch and cooperating with a projection formed on the side wall of the first container.

Preferably, it is intended that the sample holder is arranged at the closure.

Preferably, a retaining element is provided to fix the first container to the second container when it is disposed in the second receiving chamber. In particular, the retaining element may include a bayonet catch. In this context, it is preferred when the bayonet catch comprises a projection on the outside of the first container which is receivable in a recess formed in the side wall of the second container. The retaining element can also include a screw connection between the first and second containers.

Preferably, it is provided that the bottom portion of the first container is formed by a separate element sealingly abutting the side wall. This contributes to simple manufacturability and enables cost-effective manufacture by injection moulding. In particular, the separate element may include a plug. Furthermore, it is preferable for the separate element to have a sealing portion for tightly fitting into the inside of the second container. Preferably, the sealing portion can be designed as a sealing bead.

However, the bottom portion and the side wall of the first container may also be formed as one piece. In this way, the first container can be manufactured by injection moulding in one single process step.

Preferably, the second container has, at least in one portion, an elastic material pierceable with a syringe. It is preferable for the portion with the pierceable material to be located in the bottom portion. The manufacture of the second container thus formed is particularly simple when the bottom portion is a separate part connected to the side wall of the first container. In this way, the suspension solution can be safely and hygienically removed from the second container with a syringe. In this case, it is not necessary to remove the first container.

Preferably, it is intended that the filter is arranged in the bottom portion of the first container.

It is preferable for the filter to have an element tapered in a direction away from the bottom portion. In particular, the tapering element may be conical or pyramidal in shape.

Preferably, the bottom portion of the first container is provided with a base and a retaining portion for the filter.

Preferably, the retaining portion is configured so that the upper end of the filter is spaced apart from the base. Preferably, a ring-shaped space is formed by the retaining portion and the base. This improves the passage of suspension liquid through the filter.

Preferably, it is provided for the device and the method to be configured for the execution of a diagnostic test.

Preferably, it is provided that the device is designed as a stool sampling device.

It is preferable for the first and second containers to comprise plastic parts manufactured by injection moulding. In addition, it is advantageous if the sample holder and the closure are also plastic parts manufactured by injection moulding.

The device preferably comprises a further closure for liquid-tight sealing of the second container. In this way, the second container can be closed after the first container has been removed. The further closure can also be equipped with a bayonet catch.

Preferably, the liquid is introduced into the first receiving chamber prior to the sample.

A ring-shaped retaining portion projecting outwards is preferably provided on the first container. This facilitates handling and can be used as a pull-out aid when the first container is moved out of the second container. In this case, it is particularly preferred for the ring-shaped portion to have a knurling pointing outwards in a radial direction, which can easily be gripped by a user. Furthermore, the protruding ring-shaped portion may have a fluting on its side facing the second container.

In a preferred embodiment, it is provided that a total length of the first container is more than 50% greater than a total length of the second container.

Preferably, the bottom portion of the first container has a conical outer contour. Further, it is preferred when the bottom portion of the second container has a conical inner contour, wherein the conical outer contour is disposed opposite the conical inner contour when the first container is inserted into the second container. In this way, a particularly efficient sample material yield is achieved.

Further aims, characteristics, advantages and possible applications of this invention result from the following description of exemplary embodiments and the drawing. All described and/or depicted characteristics, individually or in any meaningful combination, form the object of the invention, also independently of the summary in individual claims or their back references.

The figures show:

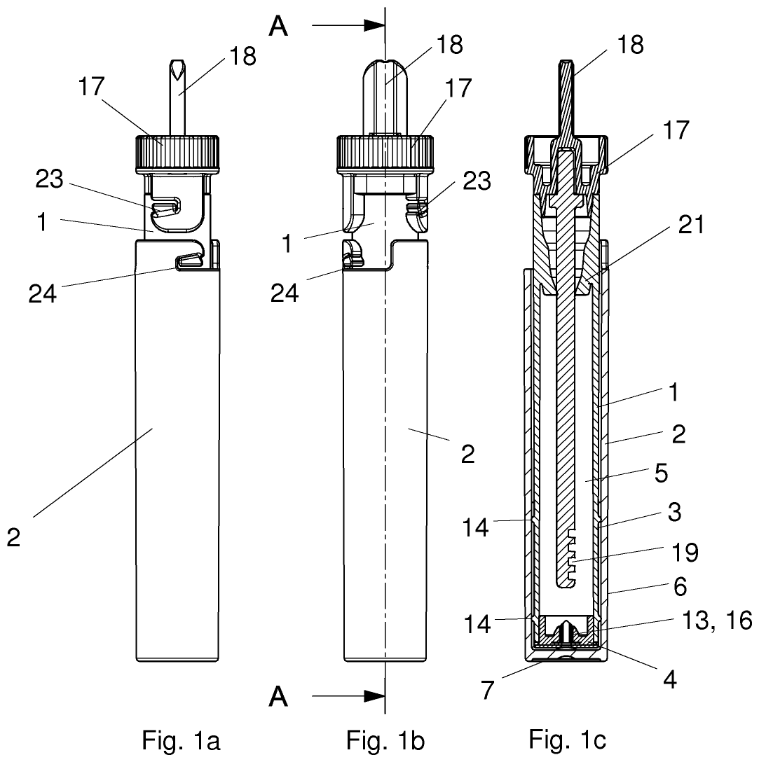

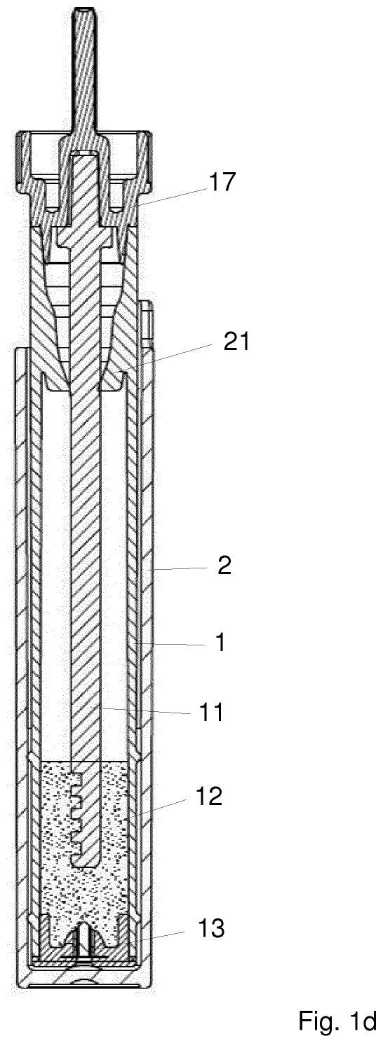

FIG. 1a: shows a device according to the invention from a first side;

FIG. 1b: shows the device from FIG. 1a from a second side;

FIG. 1c: shows the device from FIG. 1b in longitudinal section along the line A-A from FIG. 1b;

FIG. 1d: shows the device from FIG. 1c with suspension liquid and with a sample holder that is arranged rotated by 180°;

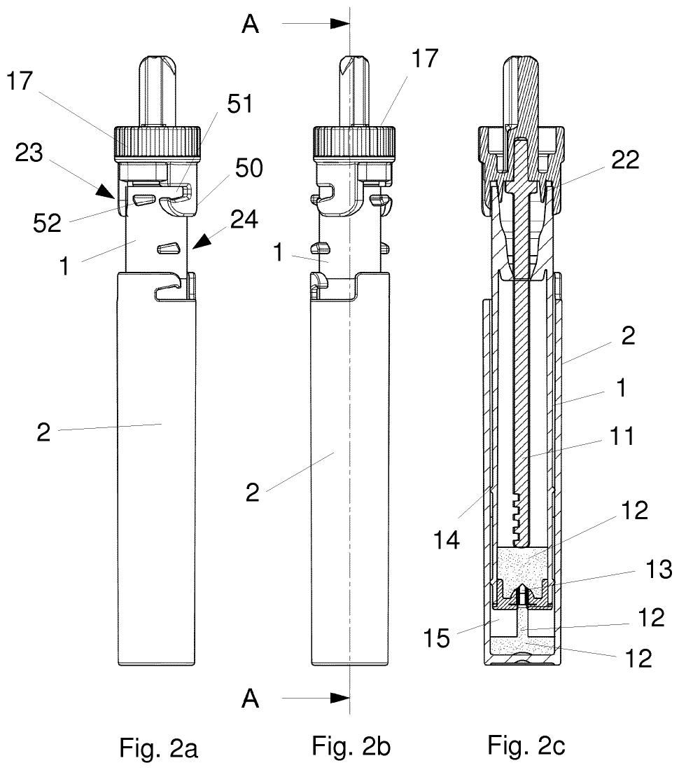

FIG. 2a: shows the device from FIG. 1d with a partially pulled out first container from a first side;

FIG. 2b: shows the container from FIG. 2a from a second side;

FIG. 2c: shows the container from FIG. 2b in longitudinal section along the line A-A;

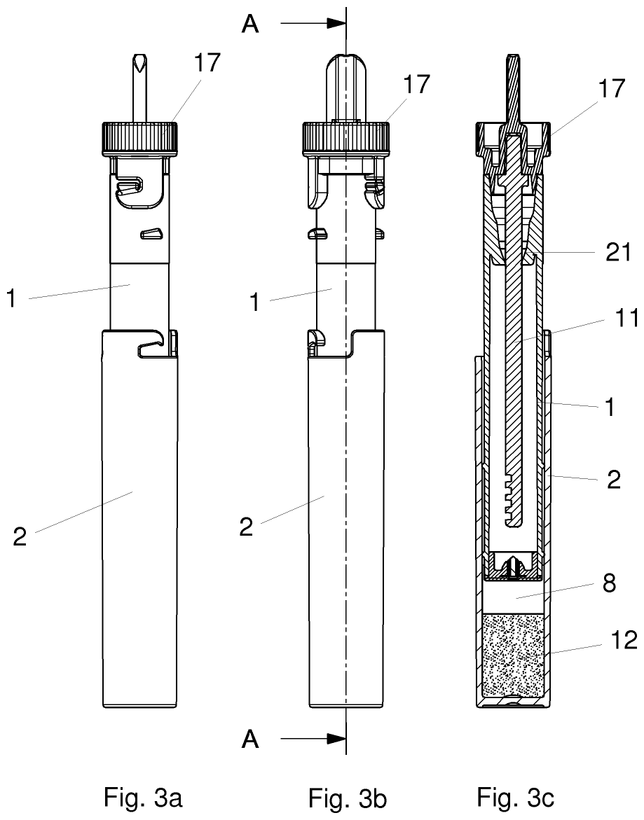

FIG. 3a: shows the device from FIG. 2a with an even further partially pulled out first container from a first side;

FIG. 3b: shows the container from FIG. 3a from a second side;

FIG. 3c: shows the container from FIG. 3b in longitudinal section along the line A-A;

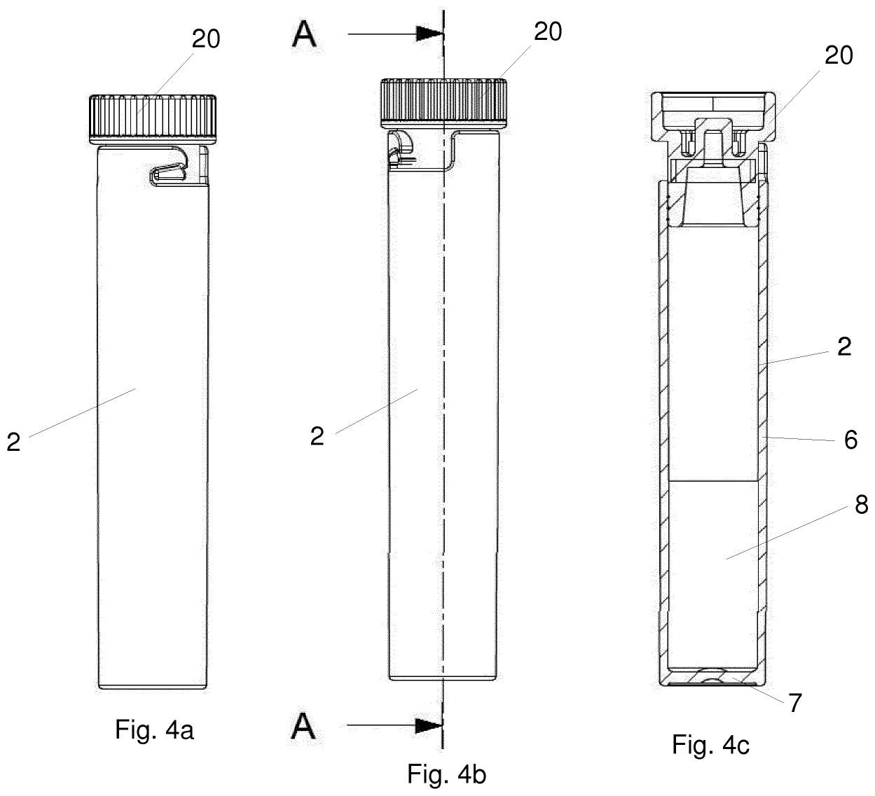

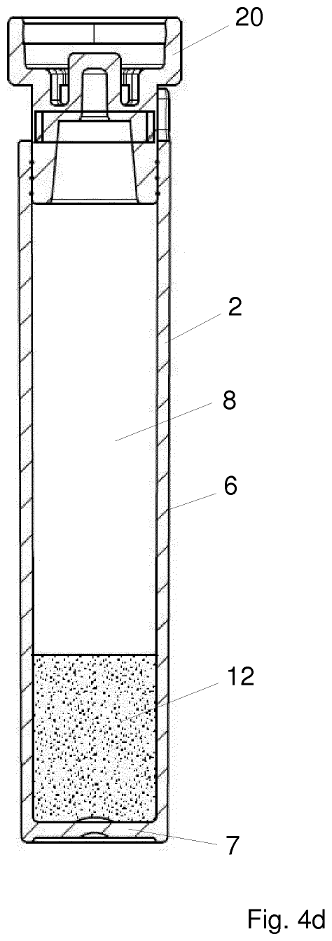

FIG. 4a: shows the second container of the device from FIG. 1a with the further closure from a first side;

FIG. 4b: shows the container from FIG. 4a from a second side;

FIG. 4c: shows the container from FIG. 4b in a longitudinal section along the line A-A;

FIG. 4d: shows the container from FIG. 4c with suspension liquid;

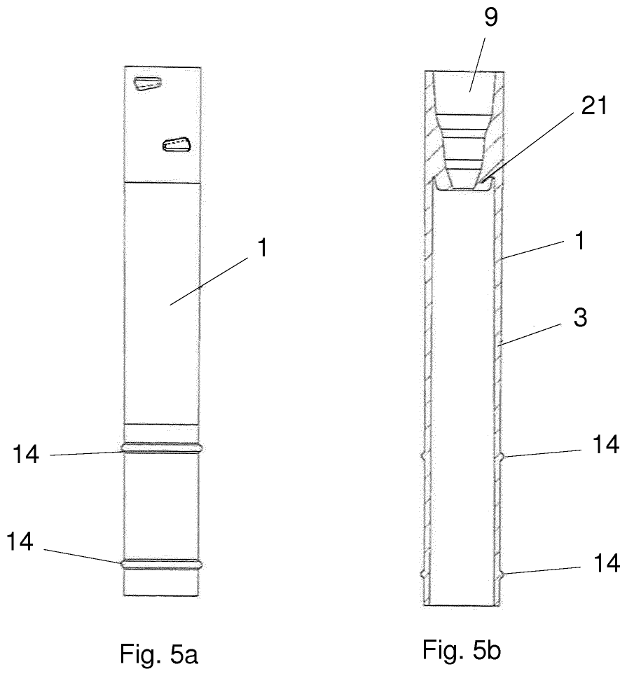

FIG. 5a: shows the side wall of the first container of the device from FIG. 1a from the side;

FIG. 5b: shows the side wall from FIG. 5a in a longitudinal section;

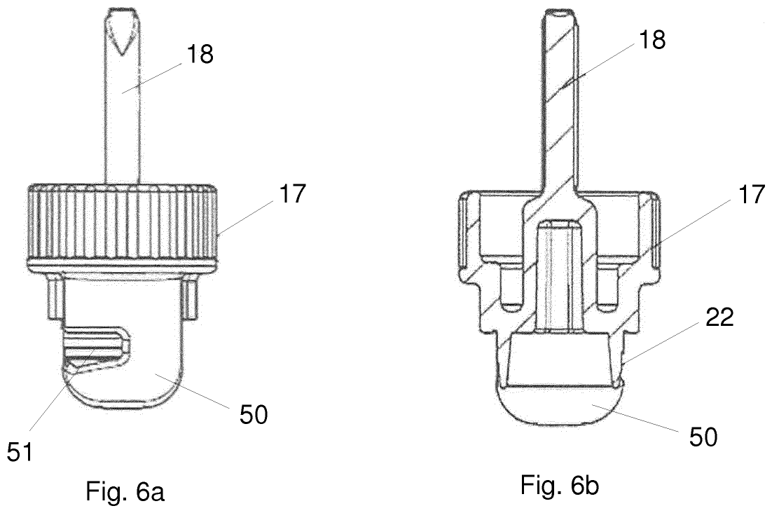

FIG. 6a: shows the closure of the first container of the device from FIG. 1a from the side;

FIG. 6b: shows the closure from FIG. 6a in a longitudinal section;

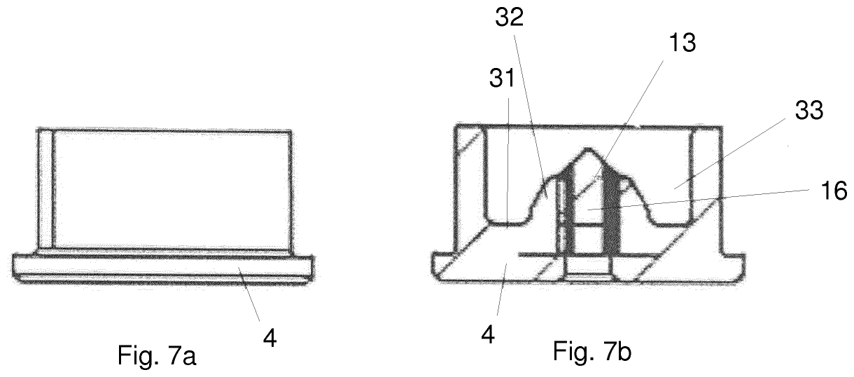

FIG. 7a: shows a separate element forming the bottom portion of the first container from FIG. 1c in a side view;

FIG. 7b: shows the separate element from FIG. 7a in longitudinal section;

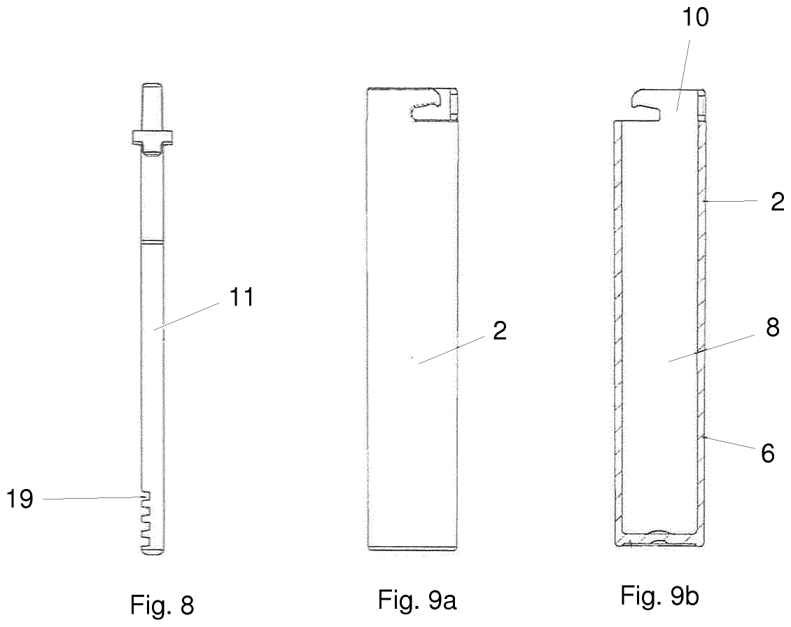

FIG. 8: shows the sample holder of the device from FIG. 1c in a side view;

FIG. 9a: shows the second container of the device from FIG. 1c in a side view;

FIG. 9b: shows the second container from FIG. 9a in a longitudinal section;

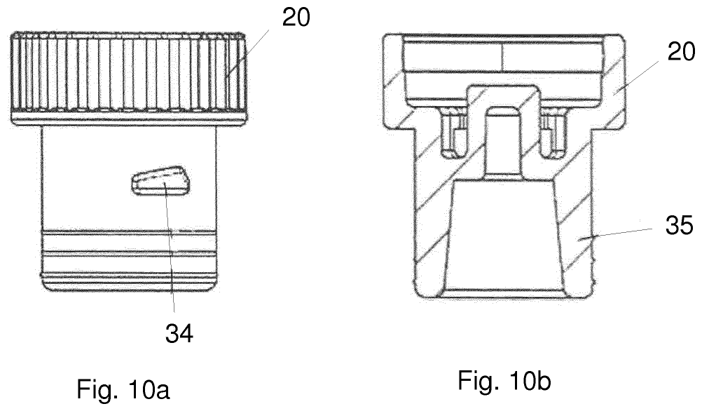

FIG. 10a: shows the further closure of the device from FIG. 4a in a side view;

FIG. 10b: shows the further closure from FIG. 10a in a longitudinal section;

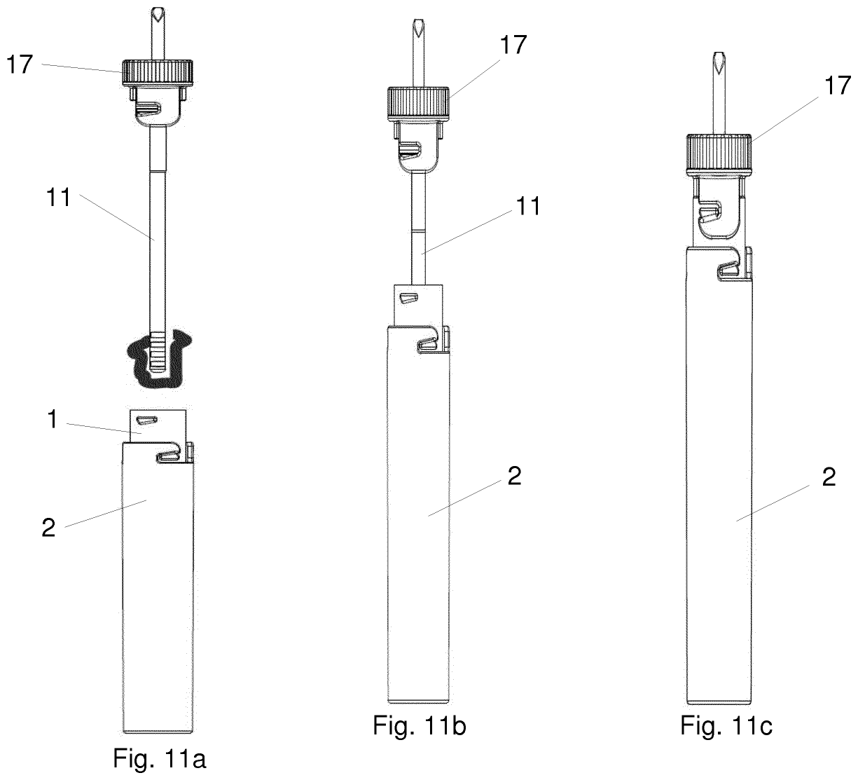

FIG. 11a-c: introducing a stool sample into the first container with the sample holder;

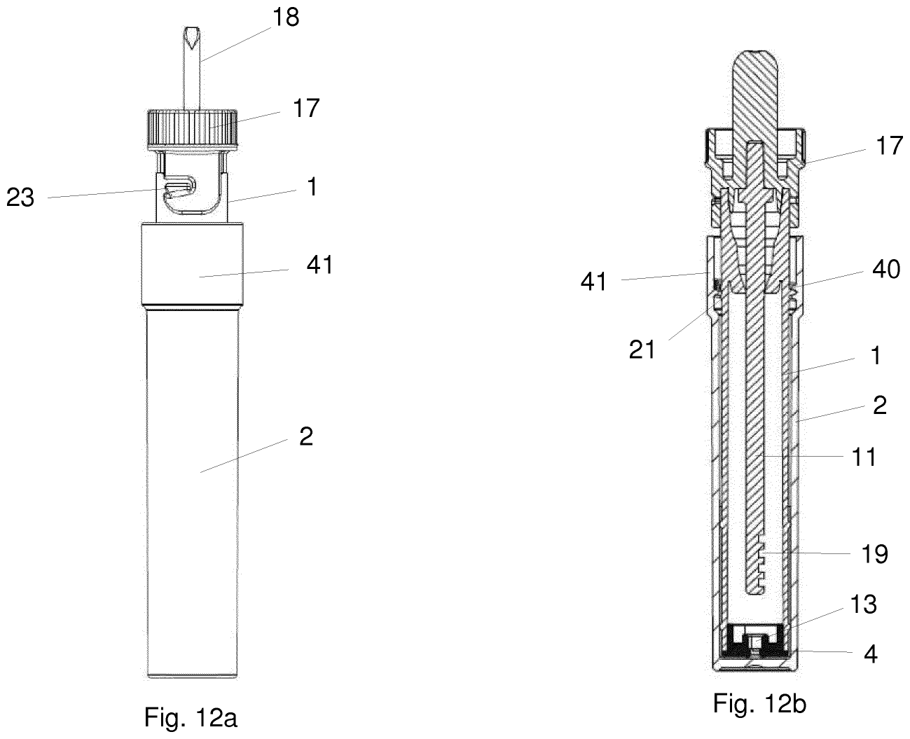

FIG. 12a-b: another embodiment of a device according to the invention from the side and in longitudinal section;

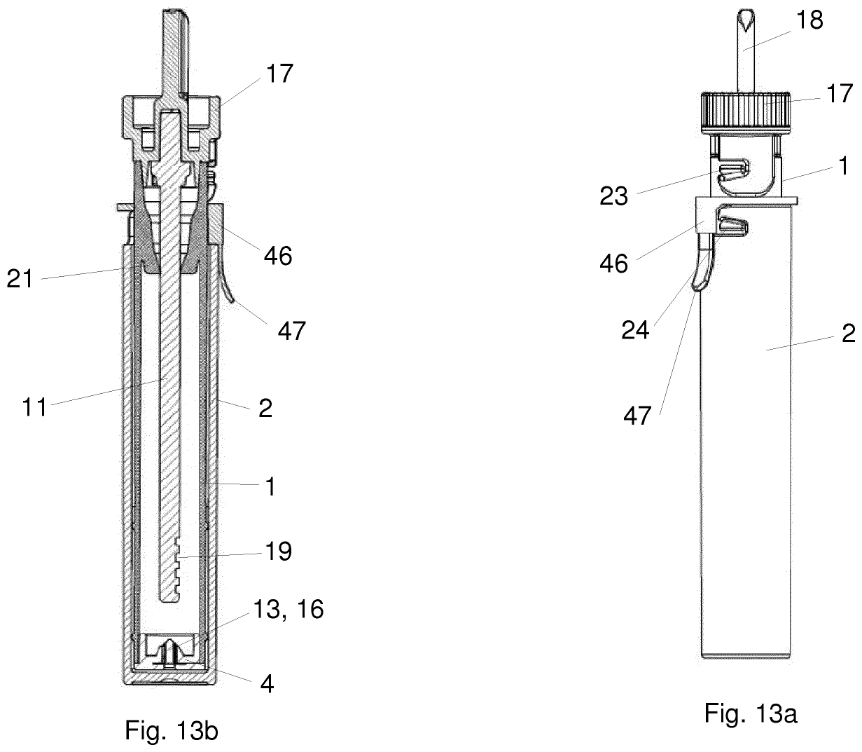

FIG. 13a-b: another embodiment of a device according to the invention from the side and in longitudinal section;

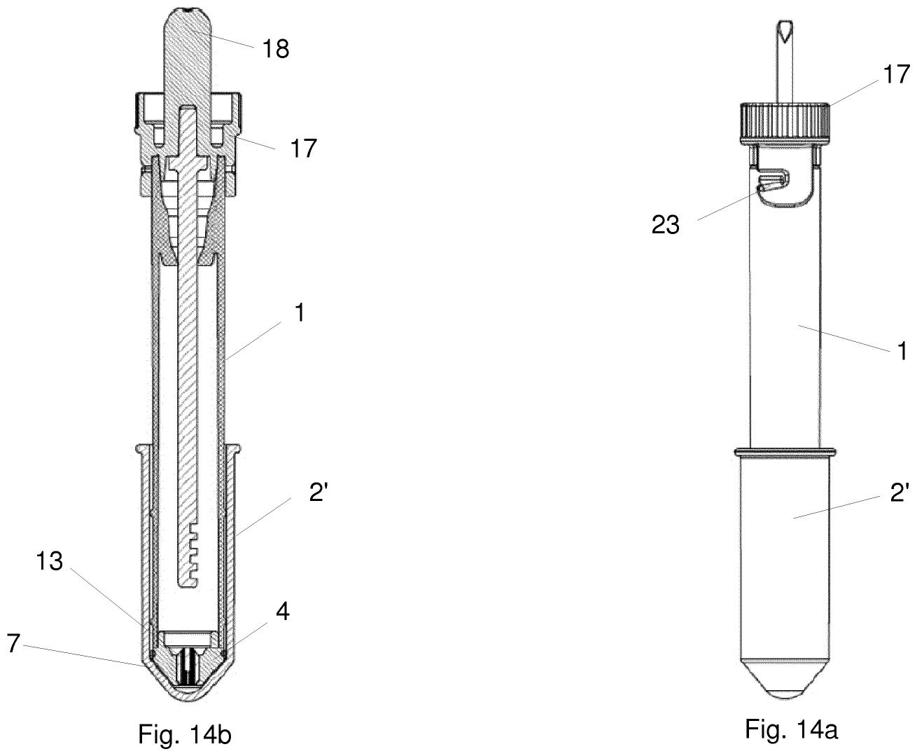

FIG. 14a-b: another embodiment of a device according to the invention from the side and in longitudinal section;

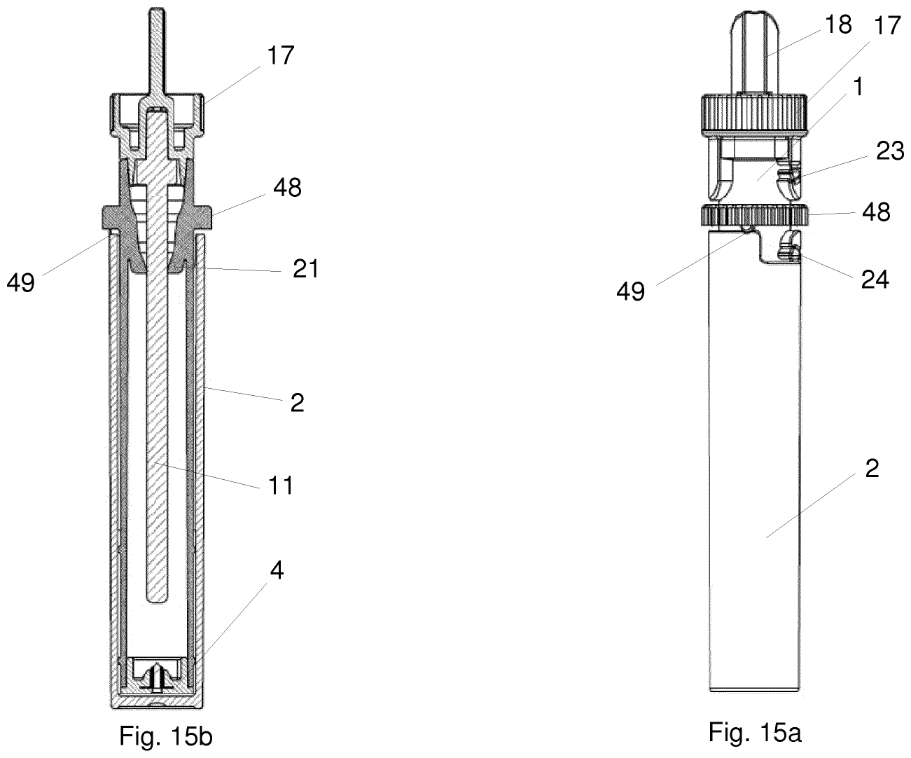

FIG. 15a-b: another embodiment of a device according to the invention from the side and in longitudinal section;

FIGS. 1-11c show a device for the preparation of a sample to be analysed comprising a first container 1 and a second container 2. The first container 1 has a side wall 3 and a bottom portion 4 which delimit a first receiving chamber 5. The second container 2 has a side wall 6 and a bottom portion 7 which delimit a second receiving chamber 8. The first container 1 has a first container opening 9 and the second container 2 a second container opening 10. The first container 1 and the second container 2 are formed in a tubular shape and each have an elongated shape. The device shown is particularly suitable for a stool sample.

As can be seen in particular from FIGS. 5a, 5b, 9a and 9b, the first container 1 and the second container 2 are separate components which can be manufactured from plastic, in particular by injection moulding. The first container 1, as shown for example in FIGS. 1a to 1c, can be arranged relocatably in the second container 2. In doing so, the first container forms an inner sleeve and the second container an outer sleeve.

The device shown also has a sample holder 11, which can be introduced into the first receiving chamber 5 with sample material as shown in FIGS. 11a-c. As shown in FIG. 1d, a suspension liquid 12 can be introduced into the first receiving chamber 5. An introduced sample can be completely or partially dissolved in the suspension liquid 12.

FIGS. 1a-3c also show that the first container 1 is fitted with a closure 17. The closure 17 has a hand grip element 18. The sample holder 11 is held at the closure 17. The sample holder 11 is arranged on the closure 17 in such a way that the sample holder 11 is located in the first receiving chamber 5 when the closure 17 is placed on the first container 1.

To prepare the sample for analysis, the first container 1 can be pulled out of the second container 2. FIGS. 2a-2c show the partially pulled out first container 1. During the pulling-out process, suspension liquid 12, which contains soluble components of the sample, is conveyed through a liquid outlet 13 arranged in the first container 1 into the second receiving chamber 8. As shown, a seal 14 is provided to seal the first container 1 against the second container 2. In this way, when the first container 1 is pulled out of the second container 2, a negative pressure in the negative pressure chamber 15 can be effectively achieved.

The first and second containers 1, 2 are thus designed in such a way that, when the first container 1 is moved out of the second receiving chamber 8, a negative pressure is created at the liquid outlet 13, so that suspension liquid can be conveyed from the first container 1 through the liquid outlet 13 into the second container 2.

FIGS. 3a-3c show the first container 1 pulled out even further from the second container 2. As in particular FIG. 3c shows, the suspension liquid 12 has now passed from the first receiving chamber 5 to the second receiving chamber 8. In doing so, unwanted particles (not shown) can be retained by the liquid outlet 13 in the first receiving chamber 5. For this purpose, the liquid outlet 13 can be equipped with a filter 16.

The filter 16 makes it possible to filter the suspension liquid as it passes through the liquid outlet 13. The filter can, for example, contain a filter material containing fibres and/or be designed as a sieve.

The figures also show that the liquid outlet is located in the bottom portion 4. As shown, bottom portion 4 is a separate component inserted into the side wall 3. In this way, the bottom portion 4 can be manufactured separately with the liquid outlet 13 and the filter 16 and then connected to the side wall 3 as shown.

FIGS. 4a-4d show the second container 2 after the complete removal of the first container 1. To prevent undesired leakage of the suspension liquid 12, the second container 2 is equipped with a removable closure 20. The closure 20 is connected to the second container 2 via a bayonet catch.

In particular, FIG. 5a shows that the seal 14 comprises circumferential projections arranged on the outside of the first container 1. In the exemplary embodiment shown, two circumferential projections are provided.

FIGS. 6a and 6b show the closure 17, which has the hand grip element 18 and the sealing element 22.

FIGS. 7a and 7b show an enlarged view of the bottom portion 4 of the first container 1. In particular, it can be easily seen that the bottom portion 4 has the liquid outlet 13 and the filter 16 arranged therein. The bottom portion 4 has a base 31 and a retaining portion 32 for the filter 16. The retaining portion 32 is configured in such a way that the upper end of the filter 16 is spaced apart from the base 4. In this way, a ring-shaped space 33 is formed by the retaining portion 32 and the base.

FIG. 8 shows that the sample holder has recesses 19 in which a defined amount of a sample is receivable.

The first container 1 is provided with a stripping device 21 which is designed as a stripping sleeve and comprises a calibration hole. The stripping device 21 shown ensures that only the recesses 19 are filled with the sample when the sample holder 11 that is provided with the sample is inserted, while excess sample material is held back at the stripping device 21 and does not get into the first receiving chamber 5.

FIGS. 9a and 9b show the embodiment of the second container 2 with the second receiving chamber 8 in detail. Furthermore, the bayonet catch for connection to the first container is clearly visible at the open end of container 1.

FIGS. 10a and 10b show an enlarged view of the closure 20 by means of which the second container 2 can be closed (see FIGS. 4a-4c). The projection 34, which allows a closure according to the bayonet principle, is clearly visible. The closure 20 has a sealing portion 35 to fit tightly into the inside of the second container 2, which enables a media-tight closure.

The closure 17 comprises a conical sealing element 22.

FIG. 2c shows that the closure 17 can assume a partially open position in which the first receiving chamber 5 is connected to the surroundings. The closure 17 has a tab 50 in which a recess 51 is arranged. The recess 51 can accommodate the projection 52 located on the outside of the first container 1 to form the bayonet catch 23.

In the FIGS. (cf. 1a and 2a in particular) the bayonet catch 23 of the first closure 17 is clearly visible. Furthermore, the further bayonet catch 24 can be seen, with which the first container 1 is attached to the second container 2.

FIGS. 7a and 7b show the bottom portion 4 of the first container, formed as a separate element. These figures also clearly show the filter 16 arranged inside.

FIGS. 11a-11c illustrate introducing a stool sample into the first container 1 with the sample holder 11; First, the sample holder 11 is brought into contact with a sample adhering to it. Then the sample holder 11 with the sample is inserted into the first container 1. The stripping device 21 (see FIG. 5b) ensures that the quantity determined by the recesses 19 of the sample holder 11 reaches the first receiving chamber 5. After the closure 17 has been placed on the first container 1 to seal it tightly, the sample can be dissolved in the suspension liquid (FIG. 11c) before the suspension liquid is conveyed to the second receiving chamber 8 as shown in FIGS. 2a-3c.

FIGS. 12a-15b show other embodiments of devices according to the invention. The exemplary embodiments shown here show essential similarities with the embodiment shown in FIGS. 1-11c. Therefore, the same reference signs as in FIGS. 1-11c are used for parts with the same function, even if the respective element is designed differently in the individual case. Moreover, in order to avoid repetitions, reference is also made to the previous figure description, which, in case of existing similarities, also applies accordingly to FIGS. 12a-15b. In the following, we emphasize the special features of the embodiments depicted in FIGS. 12a -15b.

FIGS. 12a and 12b again show a device for the preparation of a sample to be analysed, comprising a first and a second container 1, 2. In contrast to the embodiment described above, for the connection between the first container 1 and the second container 2, not a bayonet catch, but a screw connection 40 is provided. The screw connection 40 is arranged in an extended area 41 of the second container 2. The configuration of the sample holder 11 and the closure 17 is designed in exactly the same way as in the case of the embodiment described above. The same applies the to bottom portion 4 with the liquid outlet 13.

FIGS. 13a and 13b show a configuration which largely corresponds to that of FIG. 1a. However, the device shown in FIGS. 13a and 13b is additionally provided with a securing element 46. The securing element 46 is arranged on the first and second containers 1, 2 in such a way that the first container 1 can only be detached from the second container 2 and pulled out of it if the securing element 46 is first removed. In the exemplary embodiment shown, the securing element 46 blocks the bayonet catch 24 between the first and second containers 1, 2. After removing the securing element 46, the device can be operated as described in connection with FIGS. 1a-11c. The securing element 46 is designed as a securing clip in the exemplary embodiment shown. The securing element 46 is a separate component. In order to facilitate the removal of the securing element 46, it may have a predetermined breaking point. In addition, a portion of the securing element 46, as shown, may be formed as tab 47 to facilitate removal of the securing element 46. The securing element 46 can ensure that unauthorised handling of the device is detected. If the securing element 46 is present, the first container 1 cannot yet have been removed from the second container 2.

The embodiment shown in FIGS. 14a and 14b is particularly suitable for applications where only a small amount of liquid is used. The design is particularly suitable for use in laboratories. FIGS. 14a and 14b clearly show that in this embodiment the second container 2′ is formed to be considerably shorter than the first container 1. In the exemplary embodiment shown, a total length of the second container 2′ is less than 50% of a total length of the first container 1. There is also a difference in the design of the bottom portion 4 of the first container 1. The latter has a conical outer contour. In addition, the bottom portion 7 of the second container 2 has a conical outer contour. In the bottom portion 4, again the liquid outlet 13 is formed, which can be equipped with the filter 16. As shown in FIGS. 14a and 14b, the connection between the first and second containers 1, 2 may be configured without a bayonet catch.

In the embodiment shown in FIGS. 15a and 15b, the first container 1 is additionally provided with an outwardly projecting ring-shaped portion 48. This facilitates handling and can be used as a pull-out aid when the first container 1 needs to be moved out of the second container 2. As shown, the ring-shaped portion 48 has a knurling pointing outward in radial direction. At the portion 48, a projection 49 is provided for the abutting the second container 2. The projection 49 is formed as a nose. It increases the anti-rotation resistance of the first and second containers 1, 2 in relation to each other. A particularly good positioning is achieved when the second container 2 has a recess in which the projection 49 is accommodated.

What is claimed is:

1. A device for the preparation of a sample to be analysed, in particular a stool sample, comprising a first container which has a first container opening and forms a receiving chamber for a suspension liquid in which the sample is at least partially contained, a second container which has a second container opening and forms a second receiving chamber wherein the first container is at least partially receivable or received in the second receiving chamber, and a sample holder for insertion into the first container opening, wherein a liquid outlet is formed in the first container, and in that the first container and the second container are designed in such a way that suspension liquid passes from the first receiving chamber into the second receiving chamber when the first container is moved out of the second receiving chamber.

2. The device according to claim 1, wherein a seal is provided which seals the first container against the second container and wherein when the first container is pulled out of the second container, a negative pressure is created at the liquid outlet, so that the suspension liquid is conveyed from the first container through the liquid outlet into the second container.

3. The device according to claim 1, wherein the seal comprises at least one circumferential projection arranged on the first and/or the second container.

4. The device according to claim 1, wherein a filter is arranged at the liquid outlet in order to filter the suspension liquid as it passes through the liquid outlet.

5. The device according to claim 4, wherein the first container has a side wall and a bottom portion, wherein the bottom portion with the filter arranged therein is formed by a separate element which sealingly abuts the side wall

6. The device according to claim 1, wherein the sample holder has one or more recesses in which a defined quantity of the sample is receivable.

7. The device according to claim 1, wherein a stripping device for the sample holder is provided on the first container.

8. The device according to claim 1, wherein a closure is provided in order to close the first container opening in a liquid-tight manner, wherein it is possible for the closure to be brought into a partially open position in which the first receiving chamber is connected to the surroundings with the closure fitted in place.

9. The device according to claim 1, wherein a connecting means is provided for fixing the first container to the second container when it is arranged in the second receiving chamber.

10. A method for preparing a sample to be analysed, in particular a stool sample, comprising the following steps:
providing a device according to claim 1;
introducing a liquid into the first receiving chamber;
placing the sample in the first receiving chamber;
mixing the sample with the liquid to produce a suspension liquid in the first receiving chamber;
moving the first container out of the second container, wherein the suspension liquid passes through the liquid outlet from the first receiving chamber into the second receiving chamber.
